(19) United States
(12) Patent Application Publication
(10) Pub. No.: US 2026/0026893 A1
(43) Pub. Date: Jan. 29, 2026

(54) REGISTRATION FOR SPINE NAVIGATION

(71) Applicant: Medivis, Inc., New York, NY (US)

(72) Inventors: Florentin Jonas Liebmann, New York, NY (US); Long Qian, Brooklyn, NY (US); Christopher Morley, New York, NY (US); Osamah Choudhry, New York, NY (US)

(21) Appl. No.: 19/344,067

(22) Filed: Sep. 29, 2025

Related U.S. Application Data
(63) Continuation-in-part of application No. 18/244,138, filed on Sep. 8, 2023, now Pat. No. 12,456,267.
(63) Continuation-in-part of application No. 18/208,136, filed on Jun. 9, 2023.
(63) Continuation-in-part of application No. 18/380,076, filed on Oct. 13, 2023, now Pat. No. 12,462,496.
(60) Provisional application No. 63/700,752, filed on Sep. 29, 2024.

(51) Int. Cl. A61B 34/20; A61B 90/00

(52) U.S. Cl. CPC A61B 34/20; A61B 90/37; A61B 2034/2065; A61B 2034/2072; A61B 2090/3916

(57) ABSTRACT

Various embodiments of an apparatus, methods, systems and computer program products described herein are directed to a Custom Reference Engine. In some embodiments, the Custom Reference Engine captures intra-operative imagery data of a portion of physical anatomy that includes portrayal of one or more embedded fiducial markers (“embedded markers”). The Custom Reference Engine acquires a custom reference position of the portion of physical anatomy based on respective coordinates, in a unified three-dimensional (3D) coordinate space, of the one or more embedded markers. The Custom Reference Engine terminates visual interference of a view of portion of physical anatomy transforming the custom reference position over to one or more transfer fiducial markers that surround the portion of physical anatomy.

### CROSS REFERENCE TO RELATED APPLICATIONS

This application claims the benefit of priority to U.S. Provisional Application No. 63/700,752, entitled “REGISTRATION FOR SPINE NAVIGATION,” filed on Sep. 29, 2024, the entirety of which is incorporated by reference.

This application is a continuation-in-part of U.S. patent application Ser. No. 18/244,138, entitled “AUTOMATIC REGISTRATION OF LANDMARKS FOR AUGMENTED REALITY ASSISTED SURGERY.” filed on Sep. 8, 2023, the entirety of which is incorporated by reference.

This application is a continuation-in-part of U.S. patent application Ser. No. 18/208,136, entitled “SURGICAL NAVIGATION TRAJECTORY IN AUGMENTED REALITY DISPLAY,” filed on Jun. 9, 2023, the entirety of which is incorporated by reference.

This application is a continuation-in-part of U.S. patent application Ser. No. 18/380,076, entitled “DETACHED VISUALIZATION FOR SURGICAL NAVIGATION IN MIXED REALITY,” filed on Oct. 13, 2023, the entirety of which is incorporated by reference.

### BACKGROUND

Current conventional systems have limitations with regard to two-dimensional (2D) and three-dimensional (3D) images in surgical settings. Surgical planning and surgical navigation are necessary for every medical procedure. A surgeon and their team must have a plan for a case before entering an operating room, not just as a matter of good practice but to minimize malpractice liabilities and to enhance patient outcomes. Surgical planning is often conducted based on medical images including DICOM scans (MRI, CT, etc.), requiring the surgeon to flip through numerous views/slices, and utilizing this information to imagine a 3D model of the patient so that the procedure may be planned. Accordingly, in such a scenario, the best course of action is often a surgeon's judgment call based on the data that they are provided.

### SUMMARY

Various embodiments of an apparatus, methods, systems and computer program products described herein are directed to a Custom Reference Engine. In some embodiments, the Custom Reference Engine detects one or more fiducial markers situated at and near a portion of physical anatomy. The Custom Reference Engine determines coordinates of the fiducial markers according to a unified three-dimensional (3D) coordinate space. The Custom Reference Engine further determines a current position and orientation of the portion of physical anatomy relative to known fixed distances and geometries between the fiducial markers. The Custom Reference Engine determines a display position in an Augmented Reality (AR) environment of a custom reference that tracks a current position and orientation of the portion of anatomy.

However, the current display position of the custom reference may interfere with an AR visual field that includes a view of the portion of the anatomy. Due to the visual interference, the Custom Reference Engine updates the display position of the custom reference based on coordinates of fiducial markers that surround the portion of physical anatomy. The Custom Reference Engine transfers the custom reference from its current display position to an updated display position. The updated custom reference can be displayed in the AR environment without interference of the visual field while still tracking the position and orientation of the portion of anatomy.

According to various embodiments, the Custom Reference Engine captures intra-operative imagery data of a portion of physical anatomy that includes portrayal of one or more embedded fiducial markers (“embedded markers”). For example, the portion of physical anatomy may be an exposed portion of a patient's spine.

The Custom Reference Engine acquires a custom reference position of the portion of physical anatomy based on respective coordinates, in a unified three-dimensional (3D) coordinate space, of the one or more embedded markers. The Custom Reference Engine terminates visual interference of a view of portion of physical anatomy by transforming the custom reference position over to one or more transfer fiducial markers that surround the portion of physical anatomy.

In one or more embodiments, the intra-operative imagery data is captured by the Custom Reference Engine while the one or more embedded markers are connected to a physical instrument attached to the portion of physical anatomy. When the intra-operative imagery data is generated, the embedded markers, then, are depicted in the intra-operative imagery data along with a representation of the portion of physical anatomy. Stated differently, certain portions of the intra-operative imagery data portray the embedded markers.

The Custom Reference Engine detects image data representing a respective embedded marker(s) by determining a match between embedded marker image data and predefined (i.e., known) visual characteristics of an embedded marker. The Custom Reference Engine determines an image location(s) at which portrayal of respective embedded markers occurs.

According to various embodiments, the Custom Reference Engine determines the custom reference position based on one or more fixed distances and predefined geometries associated with the one or more embedded markers relative to one or more fiducial markers on a reference array of a physical instrument attached to the portion of physical anatomy.

In one or more embodiments, the Custom Reference Engine aligns an AR rendering of the intraoperative scan with a current position and orientation of a patient by registering the coordinates of the one or more embedded markers with the embedded image locations.

In one or more embodiments, the Custom Reference Engine detects one or more transfer fiducial markers physically surrounding the portion of physical anatomy.

According to various embodiments, the Custom Reference Engine transfers, according to a transformation algorithm, the custom reference position to the respective coordinates of at least one of the transfer fiducial markers. The Custom Reference Engine determines the current position and orientation of the portion of the anatomy based on the updated custom reference position.

According to various embodiments, there may be a first set of embedded fiducial markers for a first portion of a physical instrument and a second set of embedded fiducial markers for a second portion of the physical instrument. The physical instrument may be placed proximate to the portion of physical anatomy with both of the first and second sets of embedded fiducial markers. One of the sets of embedded fiducial markers may later be removed. For example, the second set of embedded markers may be removed so that only the first set of embedded markers remain and are detected by the Custom Reference Engine. Upon removal of the second set of embedded markers, the Custom Reference Engine determines the custom reference based on coordinates of the remaining first set of embedded markers.

Further areas of applicability of the present disclosure will become apparent from the detailed description, the claims and the drawings. The detailed description and specific examples are intended for illustration only and are not intended to limit the scope of the disclosure.

### BRIEF DESCRIPTION OF THE DRAWINGS

The present disclosure will become better understood from the detailed description and the drawings, wherein:

FIG. 1A is a diagram illustrating an exemplary environment in which some embodiments may operate.

FIG. 1B is a diagram illustrating an exemplary environment in which some embodiments may operate.

FIG. 2A is a diagram illustrating an exemplary method that may be performed in some embodiments.

FIG. 2B is a diagram illustrating an exemplary method that may be performed in some embodiments.

FIG. 3 is a diagram illustrating an exemplary environment in which some embodiments may operate.

FIG. 4 is a diagram illustrating an exemplary environment in which some embodiments may operate.

FIG. 5 is a diagram illustrating an exemplary environment in which some embodiments may operate.

FIG. 6 is a diagram illustrating an exemplary environment in which some embodiments may operate.

FIG. 7 is a diagram illustrating an exemplary environment in which some embodiments may operate.

### DETAILED DESCRIPTION

In this specification, reference is made in detail to specific embodiments of the invention. Some of the embodiments or their aspects are illustrated in the drawings.

For clarity in explanation, the invention has been described with reference to specific embodiments, however it should be understood that the invention is not limited to the described embodiments. On the contrary, the invention covers alternatives, modifications, and equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well known features may not have been described in detail to avoid unnecessarily obscuring the invention.

In addition, it should be understood that steps of the exemplary methods set forth in this exemplary patent can be performed in different orders than the order presented in this specification. Furthermore, some steps of the exemplary methods may be performed in parallel rather than being performed sequentially. Also, the steps of the exemplary methods may be performed in a network environment in which some steps are performed by different computers in the networked environment.

Some embodiments are implemented by a computer system. A computer system may include a processor, a memory, and a non-transitory computer-readable medium. The memory and non-transitory medium may store instructions for performing methods and steps described herein.

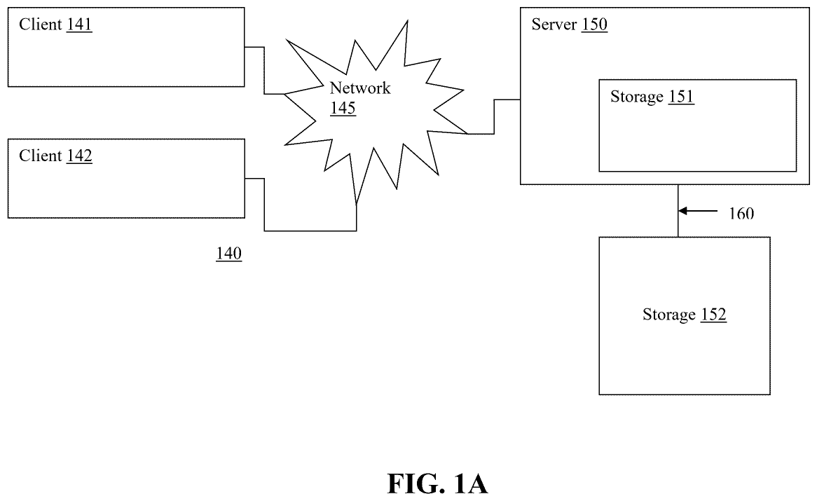

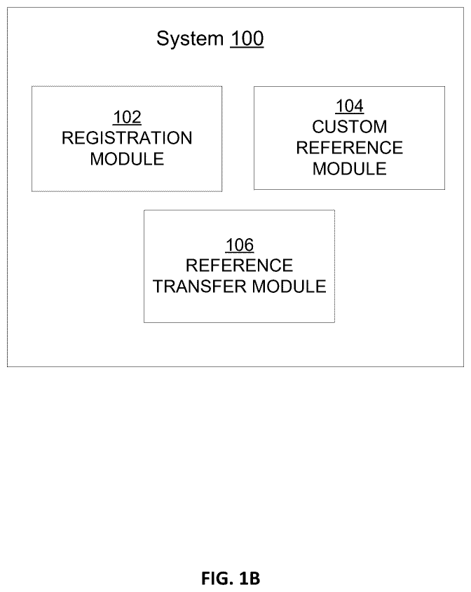

A diagram of exemplary network environment in which embodiments may operate is shown in FIG. 1A. In the exemplary environment 140, two clients 141, 142 are connected over a network 145 to a server 150 having local storage 151. Clients and servers in this environment may be computers. Server 150 may be configured to handle requests from clients.

The exemplary environment 140 is illustrated with only two clients and one server for simplicity, though in practice there may be more or fewer clients and servers. The computers have been termed clients and servers, though clients can also play the role of servers and servers can also play the role of clients. In some embodiments, the clients 141, 142 may communicate with each other as well as the servers. Also, the server 150 may communicate with other servers.

The network 145 may be, for example, local area network (LAN), wide area network (WAN), telephone networks, wireless networks, intranets, the Internet, or combinations of networks. The server 150 may be connected to storage 152 over a connection medium 160, which may be a bus, crossbar, network, or other interconnect. Storage 152 may be implemented as a network of multiple storage devices, though it is illustrated as a single entity. Storage 152 may be a file system, disk, database, or other storage.

In an embodiment, the client 141 may perform the method 200 or other method herein and, as a result, store a file in the storage 152. This may be accomplished via communication over the network 145 between the client 141 and server 150. For example, the client may communicate a request to the server 150 to store a file with a specified name in the storage 152. The server 150 may respond to the request and store the file with the specified name in the storage 152. The file to be saved may exist on the client 141 or may already exist in the server's local storage 151. In another embodiment, the server 150 may respond to requests and store the file with a specified name in the storage 151. The file to be saved may exist on the client 141 or may exist in other storage accessible via the network such as storage 152, or even in storage on the client 142 (e.g., in a peer-to-peer system).

In accordance with the above discussion, embodiments can be used to store a file on local storage such as a disk or on a removable medium like a flash drive, CD-R, or DVD-R. Furthermore, embodiments may be used to store a file on an external storage device connected to a computer over a connection medium such as a bus, crossbar, network, or other interconnect. In addition, embodiments can be used to store a file on a remote server or on a storage device accessible to the remote server.

Furthermore, cloud computing is another example where files are often stored on remote servers or remote storage systems. Cloud computing refers to pooled network resources that can be quickly provisioned so as to allow for easy scalability. Cloud computing can be used to provide software-as-a-service, platform-as-a-service, infrastructure-as-a-service, and similar features. In a cloud computing environment, a user may store a file in the “cloud,” which means that the file is stored on a remote network resource though the actual hardware storing the file may be opaque to the user.

FIG. 1B illustrates a block diagram of an example system 100 for a Custom Reference Engine that includes one or more modules. The system 100 may communicate with a user device 140 to display output, via a user interface 144 generated by an application engine. In various embodiments, the user device 140 may be an AR display headset device that further includes one or more of the respective modules 102, 104, 106 and 108.

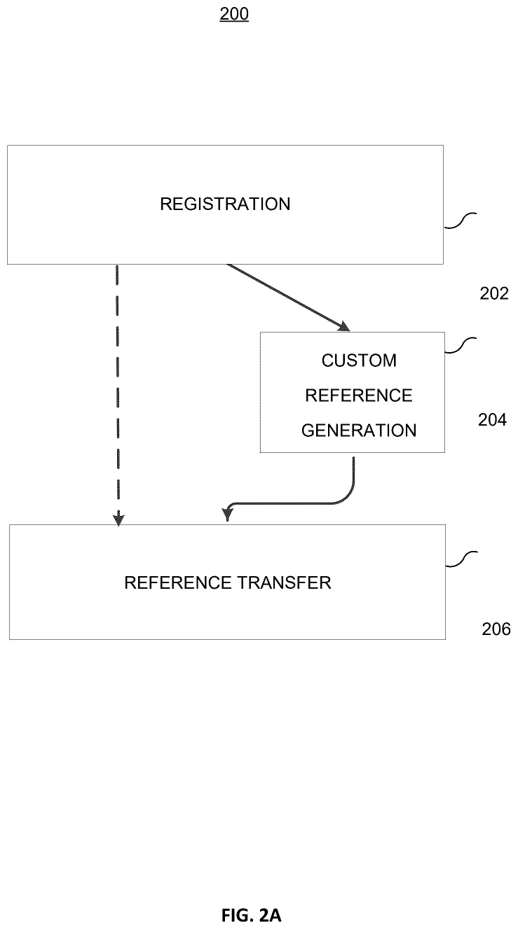

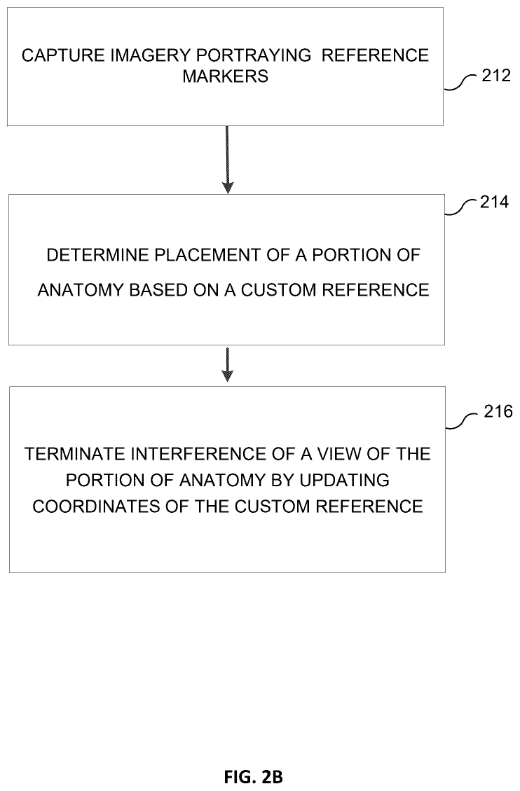

Module 102 of the system 100 may perform functionality, steps, operations, commands and/or instructions as illustrated in one or more of FIGS. 2A, 2B, 3, 4, 5, 6A, 6B (hereinafter “FIGS. 2A-6B”). In one embodiment, module 102 may perform functionality, steps, operations, commands and/or instructions related to capturing intra-operative imagery data of a portion of physical anatomy that includes portrayal of one or more embedded fiducial markers (“embedded markers”).

Module 104 of the system 100 may perform functionality, steps, operations, commands and/or instructions as illustrated in one or more of FIGS. 2A-6B. In one embodiment, module 104 may perform functionality, steps, operations, commands and/or instructions related to acquiring a custom reference position of the portion of physical anatomy based on respective coordinates, in a unified three-dimensional (3D) coordinate space, of the one or more embedded markers.

Module 106 of the system 100 may perform functionality, steps, operations, commands and/or instructions as illustrated in one or more of FIGS. 2A-6B. In one embodiment, module 106 may perform functionality, steps, operations, commands and/or instructions related to terminating visual interference of a view of portion of physical anatomy transforming the custom reference position over to one or more transfer fiducial markers that surround the portion of physical anatomy

A database associated with the system 100 maintains information, such as 3D medical model data, in a manner the promotes retrieval and storage efficiency and/or data security. In addition, the model data may include rendering parameters, such as data based on selections and modifications to a 3D virtual representation of a medical model rendered for a previous Augmented Reality display. In various embodiments, one or more rendering parameters may be preloaded as a default value for a rendering parameter in a newly initiated session of the Custom Reference Engine.

Various embodiments include an Augmented Reality (AR) headset device worn by a user which tracks one or more poses of a fiducial marker relative to one or more poses of the AR headset device. A camera(s) disposed on the AR headset device captures one or more images as the AR headset device moves. The AR headset device calculates a first spatial transformation based on the captured image(s) and a predefined fixed reference point in unified three-dimensional (3D) space (or unified 3D system) external to the AR headset device. The predefined fixed reference point represents a position in the physical world according to x, y and z coordinates. According to various embodiments, the AR headset device executes one or more simultaneous localization and mapping (SLAM) algorithms based on image input from the camera and sensor inputs. For example, sensor inputs may be data from a gyroscope or accelerometer disposed on the AR headset device. The SLAM algorithm(s) generate device pose data representing a physical orientation and a position of the AR headset device relative to the predefined fixed reference point. According to various embodiments, pose data of an instrument, fiducial marker, and/or a headset device represents a physical orientation in a 3D space defined by a unified coordinate system.

In various embodiments, the Custom Reference Engine accesses one or more storage locations that contain respective portions of medical model data. The medical model data may be represented according to two-dimensional (2D) and three-dimensional (3D) medical model data. The 2D and/or 3D (“2D/3D”) medical model data 124 may include a plurality of slice layers of medical data associated with external and internal anatomies. For example, the 2D/3D medical model data 124 may include a plurality of slice layers of medical data for generating renderings of external and internal anatomical regions of a user's head, brain and skull. It is understood that various embodiments may be directed to generating displays of any internal or external anatomical portions of the human body and/or animal bodies. In some embodiments, 2D/3D medical model data may be accessible and portrayed via a 3D cloud point representation of an anatomical region. The medical model data 124 may further be based on medical scan data.

It is understood that embodiments of the Custom Reference Engine described herein are not limited to the facial area or a head region. That is, the target portion of physical anatomy may be any anatomical portion, for example, such as a hand, leg, torso, and/or shoulder. The corresponding machine learning algorithms thereby may be, respectively, a hand landmark detection algorithm, a leg landmark detection algorithm, a torso landmark detection algorithm and/or a shoulder landmark detection algorithm.

Various embodiments described herein provide functionality for selection of menu functionalities and positional display coordinates. For example, the Custom Reference Engine tracks one or more physical gestures such as movement of a user's hand(s) and/or movement of a physical instrument(s) via one or more tracking algorithms to determine directional data to further be utilized in determining whether one or more performed physical gestures indicate a selection of one or more types of functionalities accessible via the AR display and/or selection and execution of a virtual interaction(s). For example, the Custom Reference Engine may track movement of the user's hand that results in movement of a physical instrument and/or one or more virtual offsets and virtual objects associated with the physical instrument. The Custom Reference Engine may determine respective positions and changing positions of one or more hand joints or one or more portions of the physical instrument. In various embodiments, the Custom Reference Engine may implement a simultaneous localization and mapping (SLAM) algorithm.

The Custom Reference Engine may generate directional data based at least in part on average distances between the user's palm and the user's fingers and/or hand joints or distances between portions (physical portions and/or virtual portions) of a physical instrument. In some embodiments, the Custom Reference Engine generates directional data based on detected directional movement of the AR headset device worn by the user. The Custom Reference Engine determines that the directional data is based on a position and orientation of the user's hand(s) (or the physical instrument) that indicates a portion(s) of a 3D virtual object with which the user seeks to select and/or virtually interact with and/or manipulate.

According to various embodiments, the Custom Reference Engine may implement a collision algorithm to determine a portion of a virtual object the user seeks to select and/or virtually interact with. For example, the Custom Reference Engine may track the user's hands and/or the physical instrument according to respective positional coordinates in the unified 3D coordinate system that correspond to the orientation of the user's hands and/or the physical instrument in the physical world. The Custom Reference Engine may detect that one or more tracked positional coordinates may overlap (or be the same as) one or more positional coordinates for displaying a particular portion(s) of a virtual object. In response to detecting the overlap (or intersection), the Custom Reference Engine determines that the user seeks to select and/or virtually interact with the portion(s) of the particular virtual object displayed at the overlapping positional coordinates.

According to various embodiments, upon determining the user seeks to select and/or virtually interact with a virtual object, the Custom Reference Engine may detect one or more changes in hand joint positions and/or physical instrument positions and identify the occurrence of the position changes as a performed selection function. For example, a performed selection function may represent an input command to the Custom Reference Engine confirming the user is selecting a portion of a virtual object via a ray casting algorithm and/or collision algorithm. For example, the performed selection function may also represent an input command to the Custom Reference Engine confirming the user is selecting a particular type of virtual interaction functionality. For example, the user may perform a physical gesture of tips of two fingers touching to correspond to a virtual interaction representing an input command, such as a select input command.

The Custom Reference Engine identifies one or more virtual interactions associated with the detected physical gestures. In various embodiments, the Custom Reference Engine identifies a virtual interaction selected by the user, or to be performed by the user, based on selection of one or more functionalities from a 3D virtual menu displayed in the AR display. In addition, the Custom Reference Engine identifies a virtual interaction selected by the user according to one or more pre-defined gestures that represent input commands for the Custom Reference Engine. In some embodiments, a particular virtual interaction may be identified based on a sequence of performed physical gestures detected by the Custom Reference Engine. In some embodiments, a particular virtual interaction may be identified as being selected by the user based on a series of preceding virtual interactions.

As shown in an example flowchart 200 of FIG. 2A, the Custom Reference Engine executes instructions and performs operations for any of three stages. At step 202, the Custom Reference Engine registers a current position and orientation of a portion of physical anatomy based on captured imagery that depicts embedded fiducial markers.

At step 204, the Custom Reference Engine determines a position of a custom reference relative to the current position and orientation of a portion of physical anatomy.

At step 206, the Custom Reference Engine updates coordinates of the custom reference to make it visible in an AR display environment. The updated custom reference is rendered by the Custom Reference Engine at a display location in an AR environment whereby it does not block a view of the portion of physical anatomy.

In the alternative, according to some embodiments, step 206 may be performed directly after step 202.

As shown in an example flowchart 210 of FIG. 2B, at step 212, the Custom Reference Engine captures intra-operative imagery data of a portion of physical anatomy that includes portrayal of one or more embedded fiducial markers (“embedded markers”). The generated intra-operative imagery data comprises respective depictions of the one or more embedded markers.

The Custom Reference Engine identifies image locations in the operative imagery data where depiction(s) of the embedded markers occur. The Custom Reference Engine identifies the embedded image locations be searching for matches between various portions of the operative imagery data and predefined visual characteristics and/or attributes of the embedded markers. According to one or more embodiments, various fixed distances between the embedded markers may be known. A fixed geometry amongst the embedded markers may be known as well. For example, there may be a predefined fixed distance(s) between the embedded makers and one or more fiducial markers of a reference array of a physical instrument attached to the portion of physical anatomy. The array of fiducial markers may be part of a clamp attached to the portion of anatomy.

At step 214, the Custom Reference Engine acquires a custom reference position of the portion of physical anatomy based on respective coordinates, in the unified three-dimensional (3D) coordinate space, of the one or more embedded markers. The Custom Reference Engine calculates a reference distance between the known geometry and coordinates of the embedded markers and the coordinates of the array of fiducial markers. The calculated reference distance represents a placement of the embedded markers relative to a current position and orientation of the portion of physical anatomy. The Custom Reference Engine determines the custom reference position as situated at respective coordinates of a terminus of the calculated reference distance.

At step 216, the Custom Reference Engine terminates visual interference of a view of portion of physical anatomy transforming the custom reference position over to one or more transfer fiducial markers that surround the portion of physical anatomy. The Customer Reference Engine detects one or more transfer fiducial markers physically surrounding the portion of physical anatomy. The Customer Reference Engine determines respective coordinates of the one or more transfer fiducial markers, according to the unified 3D space, in relation to the custom reference position.

The Customer Reference Engine transfers, according to a transformation algorithm, the custom reference position to the respective coordinates of at least one of the transfer fiducial markers. The Customer Reference Engine determines the current position and orientation of the portion of the anatomy based on the updated custom reference position.

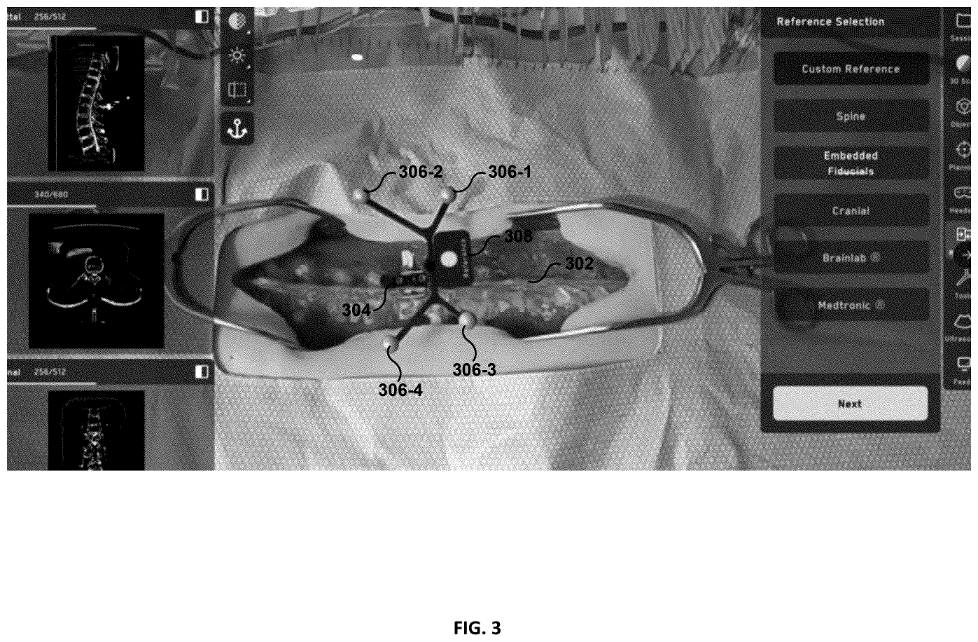

As shown in FIG. 3, a view 300 of an exposed portion of a patient's spine 302 may be provided in an AR display environment generated by an AR headset device. A first physical instrument is attached to a portion of a patient's physical anatomy. For example, the physical instrument may include a clamp portion for attaching to an exposed portion of the patient's spine 302. The clamp may have one or more fiducial markers. For example, the clamp may include an array of fiducial markers (“clamp fiducial markers”) 304. It is understood that embodiments described herein are not limited to a clamp and may include any type of instrument that can be temporarily placed or attached to any portion of a physical anatomy.

A second physical instrument may be further attached to the clamp. For example, the second physical instrument may be an array of one or more embedded fiducial markers 306-1, 306-2, 306-3, 306-4. A fixed distance between each reference fiducial marker is known 306-1, 306-2, 306-3, 306-4. Also, a fixed distance between each reference fiducial marker 306-1, 306-2, 306-3, 306-4 and the fiducial marker array 304 may be known as well. Upon attaching the second instrument to the clamp, the one or more embedded fiducial markers 306-1, 306-2, 306-3, 306-4. are situated proximate to and above the exposed portion of the patient's spine 302 to which the clamp is attached.

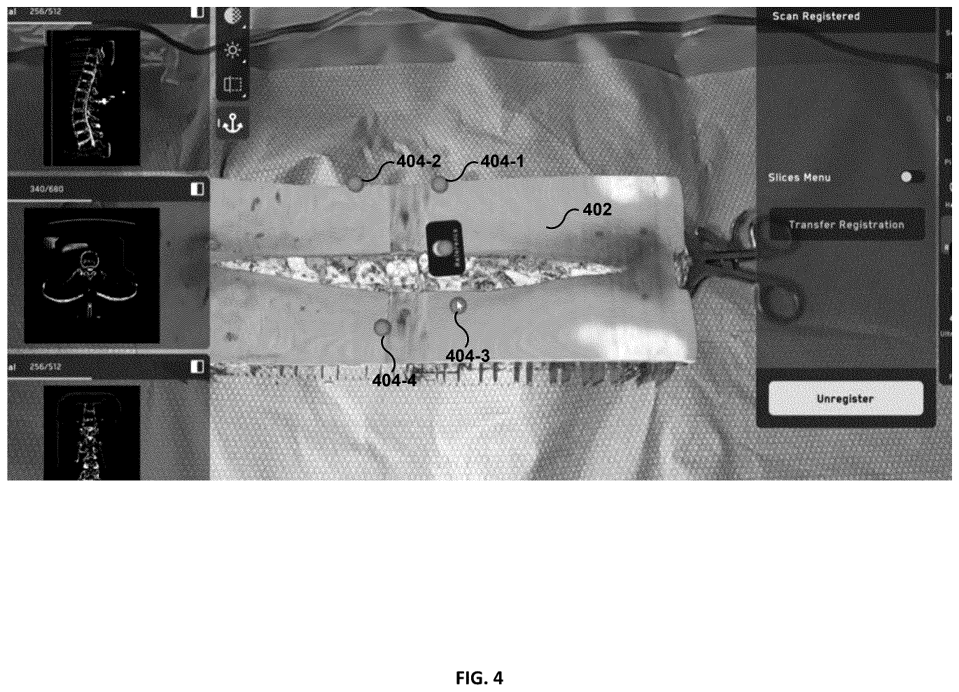

As shown in FIG. 4, the Custom Reference Engine captures an intraoperative scan of the exposed portion of the spine 302 while the clamp is attached to it and also while the second physical instrument with the embedded fiducial markers 306-1, 306-2, 306-3, 306-4 is connected to the clamp. The scan captured by the Custom Reference Engine results in generation of intraoperative image data the Custom Reference Engine may render in a view 400 of the AR display environment. The intraoperative image data includes portrayal the embedded fiducial markers combined with portrayal of medical data. The medical data corresponds to a three-dimensional (3D) representation 402 of the exposed portion of the spine in the view 400 of the AR display environment. The embedded fiducial markers may be portrayed as virtual objects 404-1, 402-2, 404-3, 404-4 in the view 400 along with a rendering of the 3D representation 402 of the exposed portion of the spine.

The embedded fiducial markers portrayed as virtual objects (“virtual reference points”) provide a reference for the actual position and orientation of the exposed portion of the spine 302. The position and orientation of each embedded fiducial markers can be determined by the Custom Reference Engine as coordinates in the unified 3D coordinate system. The coordinates of each virtual reference point 404-1, 402-2, 404-3, 404-4 may further be determined according to a position and orientation of an AR headset device worn by a user that provides a perspective view 400 of the exposed portion of the spine 302 and the AR display environment. The determined coordinates of each virtual reference point 404-1, 402-2, 404-3, 404-4 thereby become the display positions of the virtual reference points in the AR environment.

Because fixed distances between the embedded fiducial markers 306-1, 306-2, 306-3, 306-4 are known and because the fixed distances between the embedded fiducial markers 306-1, 306-2, 306-3, 306-4 and the clamp fiducial markers (such as a fiducial marker array 304) are also known, the Custom Reference Engine determines a custom reference 308 to a current position and orientation, in the unified 3D coordinate system, of the exposed portion of the spine. The Custom Reference Engine determines coordinates for the custom reference 308 relative to the coordinates for each virtual reference point 404-1, 402-2, 404-3, 404-4 and coordinates for the fiducial marker array 304.

Keeping the first and second physical instruments in their respective current physical placements may obstruct any medical professional attempting to perform a medical procedure on the exposed portion of the spine 302. It is necessary to remove the first and second physical instruments and transfer the virtual reference points to different display positions in the AR environment. However, transfer of the custom reference 308 to a different display position still requires that tracking and capturing of the current position and orientation of the exposed portion of the spine 302 can be maintained after the custom reference 308 is updated according to new coordinates.

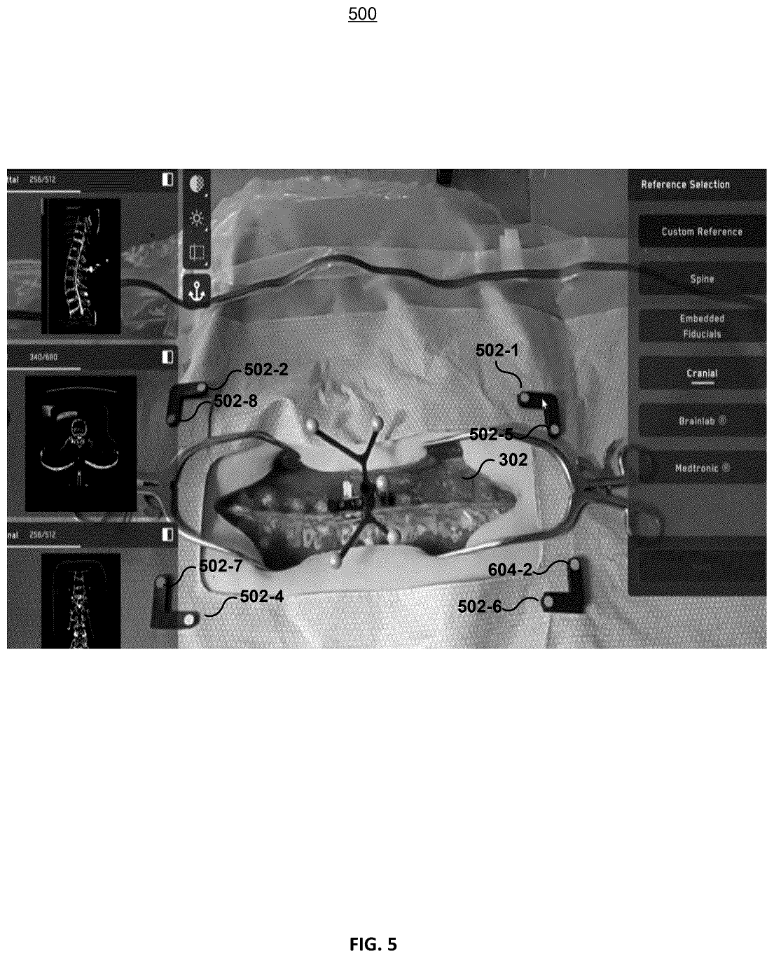

As shown in FIG. 5, one or more transfer fiducial markers (“transfer markers”) 502-1, 502-2, 502-3, 502-4, 502-5, 502-6, 502-7, 502-8 (“502”) may be situated near the exposed portion of the spine 302. The transfer markers 502 may be visible in a perspective view 500 of the AR display environment. For example, the transfer markers 502 may physically surround the exposed portion of the spine 302. The position and orientation of the transfer markers 502 are tracked and captured via the AR headset device.

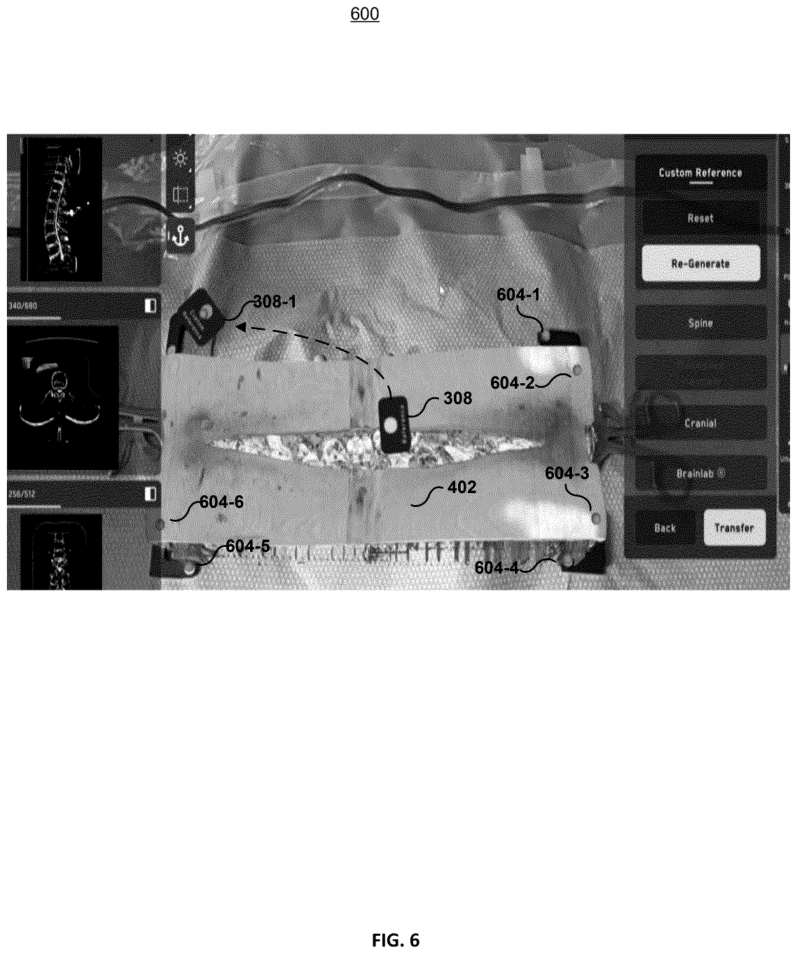

Coordinates, in the unified 3D coordinate system, for each of the transfer markers 502 are determined by the Custom Reference Engine as a result of capturing and tracking the transfer markers transfer markers 502. As shown in FIG. 6A, the Custom Reference Engine displays one or more virtual transfer reference points 604-1, 604-2, 604-3, 604-4, 604-5, 604-6 (“604”), each one representing a respective transfer marker 502, in a perspective view 600 of AR environment based on the coordinates.

The Custom Reference Engine implements a transformation algorithm according to one or more of the virtual reference points 404-1, 402-2, 404-3, 404-4. The Custom Reference Engine's performance of the transformation algorithm transfers the current coordinates for the custom reference 308 over to one of the virtual transfer reference points 604. For example, an updated position and orientation can be determined in reference to the coordinates for the virtual reference points. Since coordinates for the virtual transfer reference points 604 (corresponding to the transfer markers 502) are known and tracked, the transformation algorithm determines the distance, position and orientation of the exposed portion of the spine relative to the coordinates for the virtual transfer reference points 604.

Once the transformation algorithm is complete, the custom reference 308-1 is updated to correspond to one or more of the virtual transfer reference points 604. A current position and orientation of the exposed portion of the spine is thereby tracked and determined relative to updated coordinates of the custom reference 308-1. The first and second physical instruments can then be removed. Display of the virtual reference points in the AR environment can also be terminated.

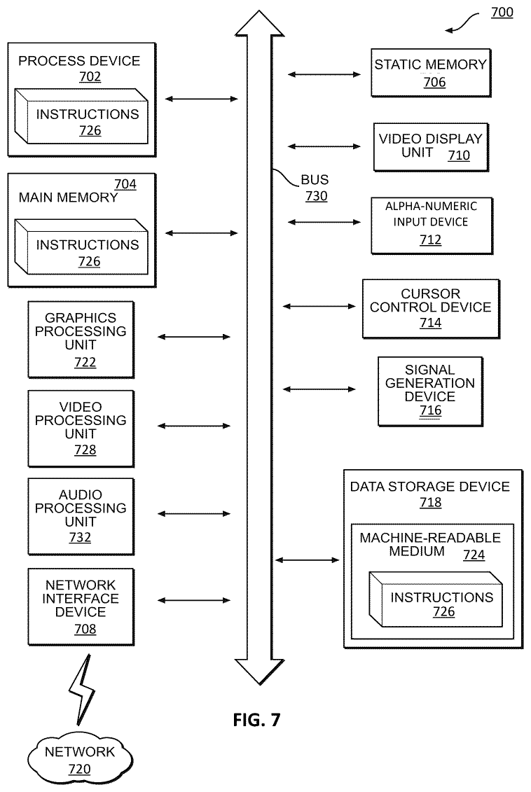

FIG. 7 illustrates an example machine of a computer system within which a set of instructions, for causing the machine to perform any one or more of the methodologies discussed herein, may be executed. In alternative implementations, the machine may be connected (e.g., networked) to other machines in a LAN, an intranet, an extranet, and/or the Internet. The machine may operate in the capacity of a server or a client machine in client-server network environment, as a peer machine in a peer-to-peer (or distributed) network environment, or as a server or a client machine in a cloud computing infrastructure or environment.

The machine may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The example computer system 700 includes a processing device 702, a main memory 704 (e.g., read-only memory (ROM), flash memory, dynamic random access memory (DRAM) such as synchronous DRAM (SDRAM) or Rambus DRAM (RDRAM), etc.), a static memory 706 (e.g., flash memory, static random access memory (SRAM), etc.), and a data storage device 718, which communicate with each other via a bus 730.

Processing device 702 represents one or more general-purpose processing devices such as a microprocessor, a central processing unit, or the like. More particularly, the processing device may be complex instruction set computing (CISC) microprocessor, reduced instruction set computing (RISC) microprocessor, very long instruction word (VLIW) microprocessor, or processor implementing other instruction sets, or processors implementing a combination of instruction sets. Processing device 702 may also be one or more special-purpose processing devices such as an application specific integrated circuit (ASIC), a field programmable gate array (FPGA), a digital signal processor (DSP), network processor, or the like. The processing device 702 is configured to execute instructions 726 for performing the operations and steps discussed herein.

The computer system 700 may further include a network interface device 708 to communicate over the network 720. The computer system 700 also may include a video display unit 710 (e.g., a liquid crystal display (LCD) or a cathode ray tube (CRT)), an alphanumeric input device 712 (e.g., a keyboard), a cursor control device 714 (e.g., a mouse), a graphics processing unit 722, a signal generation device 716 (e.g., a speaker), graphics processing unit 722, video processing unit 728, and audio processing unit 732.

The data storage device 718 may include a machine-readable storage medium 724 (also known as a computer-readable medium) on which is stored one or more sets of instructions or software 726 embodying any one or more of the methodologies or functions described herein. The instructions 726 may also reside, completely or at least partially, within the main memory 704 and/or within the processing device 702 during execution thereof by the computer system 700, the main memory 704 and the processing device 702 also constituting machine-readable storage media.

In one implementation, the instructions 726 include instructions to implement functionality corresponding to the components of a device to perform the disclosure herein. While the machine-readable storage medium 724 is shown in an example implementation to be a single medium, the term “machine-readable storage medium” should be taken to include a single medium or multiple media (e.g., a centralized or distributed database, and/or associated caches and servers) that store the one or more sets of instructions. The term “machine-readable storage medium” shall also be taken to include any medium that is capable of storing or encoding a set of instructions for execution by the machine and that cause the machine to perform any one or more of the methodologies of the present disclosure. The term “machine-readable storage medium” shall accordingly be taken to include, but not be limited to, solid-state memories, optical media and magnetic media.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “identifying” or “determining” or “executing” or “performing” or “collecting” or “creating” or “sending” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

Various general purpose systems may be used with programs in accordance with the teachings herein, or it may prove convenient to construct a more specialized apparatus to perform the method. The structure for a variety of these systems will appear as set forth in the description above. In addition, the present disclosure is not described with reference to any particular programming language. It will be appreciated that a variety of programming languages may be used to implement the teachings of the disclosure as described herein.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a machine (e.g., a computer). For example, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

In the foregoing disclosure, implementations of the disclosure have been described with reference to specific example implementations thereof. It will be evident that various modifications may be made thereto without departing from the broader spirit and scope of implementations of the disclosure as set forth in the following claims. The disclosure and drawings are, accordingly, to be regarded in an illustrative sense rather than a restrictive sense.

What is claimed is:

1. A computer-implemented method, comprising:
capturing intra-operative imagery data of a portion of physical anatomy that includes portrayal of one or more embedded fiducial markers (“embedded markers”);
acquiring a custom reference position of the portion of physical anatomy based on respective coordinates, in a unified three-dimensional (3D) coordinate space, of the one or more embedded markers; and
terminating visual interference of a view of portion of physical anatomy by transforming the custom reference position over to one or more transfer fiducial markers that surround the portion of physical anatomy.

2. The computer-implemented method of claim 1, wherein the intra-operative imagery data comprises portrayal of the one or more embedded markers.

3. The computer-implemented method of claim 1, wherein acquiring a custom reference position comprises:
determining the custom reference position based on one or more fixed distances associated with the one or more embedded markers relative to one or more fiducial markers on a reference array of a physical instrument attached to the portion of physical anatomy.

4. The computer-implemented method of claim 1, wherein transforming the custom reference position comprises:
detecting one or more transfer fiducial markers physically surrounding the portion of physical anatomy;
determining respective coordinates of the one or more transfer fiducial markers, according to the unified 3D space, in relation to the custom reference position.

5. The computer-implemented method of claim 4, further comprising:
updating the custom reference position relative to the one or more transfer fiducial markers and the current position and orientation of the portion of the anatomy.

6. The computer-implemented method of claim 5, wherein updating the custom reference position comprises:
transferring, according to a transformation algorithm, the custom reference position to the respective coordinates of at least one of the transfer fiducial markers; and
determining the current position and orientation of the portion of the anatomy based on the updated custom reference position.

7. The computer-implemented method of claim 6, wherein terminating visual interference of a view of portion of physical anatomy comprises:
initiating display of the update custom reference position at the respective coordinates of at least one of the transfer fiducial markers.

8. A system comprising one or more processors, and a non-transitory computer-readable medium including one or more sequences of instructions that, when executed by the one or more processors, cause the system to perform operations comprising:
capturing intra-operative imagery data of a portion of physical anatomy that includes portrayal of one or more embedded fiducial markers (“embedded markers”);
acquiring a custom reference position of the portion of physical anatomy based on respective coordinates, in a unified three-dimensional (3D) coordinate space, of the one or more embedded markers; and
terminating visual interference of a view of portion of physical anatomy by transforming the custom reference position over to one or more transfer fiducial markers that surround the portion of physical anatomy.

9. The system of claim 8, wherein the intra-operative imagery data comprises portrayal of the one or more embedded markers.

10. The system of claim 8, wherein acquiring a custom reference position comprises:
determining the custom reference position based on one or more fixed distances associated with the one or more embedded markers relative to one or more fiducial markers on a reference array of a physical instrument attached to the portion of physical anatomy.

11. The system of claim 8, wherein transforming the custom reference position comprises:
detecting one or more transfer fiducial markers physically surrounding the portion of physical anatomy;
determining respective coordinates of the one or more transfer fiducial markers, according to the unified 3D space, in relation to the custom reference position.

12. The system of claim 11, further comprising:
updating the custom reference position relative to the one or more transfer fiducial markers and the current position and orientation of the portion of the anatomy.

13. The system of claim 12, wherein updating the custom reference position comprises:
transferring, according to a transformation algorithm, the custom reference position to the respective coordinates of at least one of the transfer fiducial markers; and
determining the current position and orientation of the portion of the anatomy based on the updated custom reference position.

14. The system of claim 13, wherein terminating visual interference of a view of portion of physical anatomy comprises:
initiating display of the update custom reference position at the respective coordinates of at least one of the transfer fiducial markers.

15. A computer program product comprising a non-transitory computer-readable medium having a computer-readable program code embodied therein to be executed by one or more processors, the program code including instructions for:
capturing intra-operative imagery data of a portion of physical anatomy that includes portrayal of one or more embedded fiducial markers (“embedded markers”);
acquiring a custom reference position of the portion of physical anatomy based on respective coordinates, in a unified three-dimensional (3D) coordinate space, of the one or more embedded markers; and
terminating visual interference of a view of portion of physical anatomy by transforming the custom reference position over to one or more transfer fiducial markers that surround the portion of physical anatomy.

16. The computer program product of claim 15, wherein the intra-operative imagery data comprises portrayal of the one or more embedded markers.

17. The computer program product of claim 15, wherein acquiring a custom reference position comprises:
determining the custom reference position based on one or more fixed distances associated with the one or more embedded markers relative to one or more fiducial markers on a reference array of a physical instrument attached to the portion of physical anatomy.

18. The computer program product of claim 15, wherein transforming the custom reference position comprises:
detecting one or more transfer fiducial markers physically surrounding the portion of physical anatomy;
determining respective coordinates of the one or more transfer fiducial markers, according to the unified 3D space, in relation to the custom reference position.

19. The computer program product of claim 18, further comprising:
updating the custom reference position relative to the one or more transfer fiducial markers and the current position and orientation of the portion of the anatomy.

20. The computer program product of claim 19, wherein updating the custom reference position comprises:
transferring, according to a transformation algorithm, the custom reference position to the respective coordinates of at least one of the transfer fiducial markers; and
determining the current position and orientation of the portion of the anatomy based on the updated custom reference position;
wherein terminating visual interference of a view of portion of physical anatomy comprises:
initiating display of the update custom reference position at the respective coordinates of at least one of the transfer fiducial markers.
